(19) United States
(12) Patent Application Publication
(10) Pub. No.: US 2022/0096391 A1
(43) Pub. Date: Mar. 31, 2022

(54) UNIDIRECTIONAL PRESENTATION OF MEMBRANE PROTEINS IN NANOPARTICLE-SUPPORTED LIPOSOMES

(71) Applicant: PRESIDENT AND FELLOWS OF HARVARD COLLEGE, Cambridge, MA (US)

(72) Inventors: James CHOU, Cambridge, MA (US); Wen CHEN, Cambridge, MA (US)

(73) Assignee: PRESIDENT AND FELLOWS OF HARVARD COLLEGE, Cambridge, MA (US)

(21) Appl. No.: 17/419,160

(22) PCT Filed: Dec. 26, 2019
(86) PCT No.: PCT/US2019/068601

§ 371 Date: Jun. 28, 2021

Related U.S. Application Data
(60) Provisional application No. 62/785,897, filed on Dec. 28, 2018.

(51) Int. Cl. A61K 9/51; A61K 39/21; A61K 9/127; G01N 33/543; A61K 39/00

(52) U.S. Cl. CPC A61K 9/5115; A61K 39/21; A61K 2039/55555; G01N 33/54346; C12N 2740/16234; A61K 9/1271

(57) ABSTRACT

Presentation of membrane proteins to host immune systems has been a challenging problem due to complexity arising from the poor in vivo stability of the membrane-mimetic media often used for solubilizing the membrane proteins. The Inventors report the use of functionalized, biocompatible nanoparticles as substrates to guide the formation of proteoliposomes that can present many copies of membrane proteins in a unidirectional manner. The approach was demonstrated to present the membrane-proximal region of the HIV-1 envelope glycoprotein. These nanoparticle-supported liposomes are broadly applicable as membrane antigen vehicles for inducing host immune responses. In some instances, the technology supports generation of antibodies that do not generate an immunogenic response in comparison to conventional protein presentation (i.e., liposome).

### CROSS-REFERENCE TO RELATED APPLICATIONS

This application is a 35 U.S.C. § 371 National Phase Entry Application of International Patent Application No. PCT/US2019/068601 filed on Dec. 26, 2019, which designated the U.S., which claims benefit under 35 U.S.C. § 119(e) of U.S. Provisional Application No. 62/785,897 filed Dec. 28, 2018, the contents of which are incorporated herein by reference in their entireties.

### STATEMENT REGARDING FEDERALLY-SPONSORED RESEARCH

This invention was made with government support under GM116898 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Dec. 26, 2019, is named 002806-094040WOPT_SL.txt and is 980 bytes in size.

### FIELD OF THE INVENTION

Described herein are compositions and methods related to presenting proteins in biologically relevant spatial orientations to detect and analyze specific protein features of interest.

### BACKGROUND

The large majority of antibodies currently being developed for therapeutic use target membrane-anchored or integral membrane proteins. But presenting these proteins to immune systems to induce antibodies is a difficult problem, in particular, for those whose structural integrity can only be preserved on the membrane. Several approaches have been developed to address this problem. Liposomes are often used as membrane protein carriers for inducing immune responses. During liposome formation, however, the protein orientation is random. The unstable nature of the liposome, i.e., its tendency to fuse with other cellular vesicles, could be another source of risk in its application. Greater stability in serum could be achieved using interbilayer-crosslinked multilamellar vesicle (ICMV)-coated particles, but the technique has not been demonstrated to incorporate transmembrane proteins. Lipid nanodisc is another popular medium for membrane proteins, and has been used previously in phage display. But, nanodisc samples are generally difficult to make in large quantities. Moreover, regular nanodiscs usually can only contain 1-2 copies of protein due to its small size (10-15 nm in diameter), and are thus not ideal for inducing strong immunogenic responses in vivo. It is also possible to use mammalian cells to produce virus-like particles (VLP) incorporating membrane proteins on the VLP membrane. The success of this approach, however, depends on the efficiency of protein incorporation into VLP, which needs laborious optimization for each target and often cannot be controlled manually. There is a great need in the art for unidirectional presentation of membrane proteins.

Described herein are methods and compositions for using nanoparticles as substrates to guide proteoliposome assembly. By modifying nanoparticles with functional moieties that specifically recruit affinity-tagged membrane proteins in bicelles, the Inventors could form proteoliposomes around the nanoparticles where the proteins are presented in a unidirectional manner. The Inventors have demonstrated this approach, named Supported ProteoLiposome for Antigen Directed Display (SPLAnDiD), for a membrane fragment of the HIV-1 envelope glycoprotein (Env) encompassing the transmembrane (TM) domain and the membrane-proximal external region (MPER).

### SUMMARY OF THE INVENTION

Described herein is an assembly including a substrate, a coating, a quantity of lipids including a protein with a tag, wherein the coating is attached to the substrate and bound to the tag. In other embodiments, the substrate is globular, solid, hollow, porous, multi-layer, and combinations thereof. In other embodiments, the substrate is spherical, cubic, tetrahedral, tubular, or in any three dimensional shape. In other embodiments, the substrate is a nanoparticle. In other embodiments, the nanoparticle includes a gold nanoparticle, silver nanoparticle, platinum nanoparticle, silicon dioxide nanoparticle, porous silicon nanoparticle, polymer nanoparticle, and/or complex nanoparticle. In other embodiments, the substrate is a biological molecule. In other embodiments, the biological molecule is selected from the group consisting of, DNA, RNA, PNA, LNA, and protein. In other embodiments, the DNA is a buckyball, cube, tetrahedron, dodecahedron, pyramid, tube, or stick. In other embodiments, the DNA includes a di-sulfide modifier, amino modifier, azide modifier, acrydite modifier, alkyne modifier, biotin, and/or digoxigenin. In other embodiments, the coating includes polyphenol, tannic acid, catechin, dopamine, theaflavin, anthocyanidin, and derivatives thereof. In other embodiments, the coating includes one or more molecules selected from the group consisting of, PEG-SMCC, AMAS, BMPS, GMBS, MBS, EMCS, SMPB, SMPH, SPDP, and SMPT. In other embodiments, the coating includes lysine, and/or cysteine. In other embodiments, the coating includes NTA-Ni, antibodies, nanobodies, biotin, and/or streptavidin In other embodiments, the lipids are bicelles, synthetic lipids, extracts from host, and combinations thereof. In other embodiments, the tag is one or more tags selected from the group consisting of, histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag. In other embodiments, the coating attached to the substrate and bound to the tag externally presents a feature of the protein.

Also described herein is a method, including attaching a coating to a substrate, and adding a functional moiety to the coating. In other embodiments, the substrate is globular, solid, hollow, porous, multi-layer, and combinations thereof. In other embodiments, the substrate is spherical, cubic, tetrahedral, tubular, or in any three dimensional shape. In other embodiments, the substrate is a nanoparticle. In other embodiments, the nanoparticle includes a gold nanoparticle, silver nanoparticle, platinum nanoparticle, silicon dioxide nanoparticle, porous silicon nanoparticle, polymer nanoparticle, and/or complex nanoparticle. In other embodiments, the substrate is a biological molecule. In other embodiments, the biological molecule is selected from the group consisting of, DNA, RNA, PNA, LNA, and protein. In other embodiments, the DNA is a buckyball, cube, tetrahedron, dodecahedron, pyramid, tube, or stick. In other embodiments, the DNA includes a di-sulfide modifier, amino modifier, azide modifier, acrydite modifier, alkyne modifier, biotin, and/or digoxigenin. In other embodiments, the coating includes polyphenol, tannic acid, catechin, dopamine, theaflavin, anthocyanidin, and derivatives thereof. In other embodiments, the coating includes PEG-SMCC AMAS, BMPS, GMBS, MBS, EMCS, SMPB, SMPH, SPDP, and SMPT. In other embodiments, the coating includes lysine and/or cysteine. In other embodiments, the functional moiety includes NTA-Ni, antibodies, nanobodies, biotin, and/or streptavidin. In other embodiments, the tag is one or more tags selected from the group consisting of, histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag. In other embodiments, the method includes adding a bicelle including a protein with a tag to the substrate. In other embodiments, the bicelle is attached to a detergent that is removed prior to addition. In other embodiments, the tag is attached to the functional moiety, thereby externally presenting a feature of the protein.

Further described herein is a method of detecting a protein, including, incubating a tagged protein with a lipid and a detergent to form an assembly, removing the detergent, incubating the assembly in the presence of a coated substrate, wherein the coated substrate includes a functional moiety capable of binding to the tagged protein, forming proteoliposomes on the coated substrate to externally present a feature of the tagged protein, and detecting the protein using the externally presented feature. In other embodiments, the protein is a membrane bound protein, including transmembrane protein, membrane transport protein, channel protein, membrane receptor, membrane anchored protein, and membrane protein complex.

### BRIEF DESCRIPTION OF THE FIGURES

FIG. 1A-1D. Schematic illustration of nanoparticle-supported proteoliposome for unidirectional presentation of membrane proteins. (FIG. 1a) The solid nanoparticle (e.g., polyphenol-stabilized gold nanoparticle) and the hollow DNA buckyball can both be used as core substrates for liposome formation. (FIG. 1b) Membrane proteins with cytoplasmic affinity tag are reconstituted in bicelles that mimic a lipid bilayer. (FIG. 1c) Functional moieties for interacting with the protein affinity tag are covalently linked to the nanoparticles. (FIG. 1d) High copy numbers of membrane proteins are presented in a unidirectional manner in a nanoparticle-supported liposome.

FIG. 2A-2G. Nanoparticle-supported proteoliposome formation using bicelle-reconstituted MPER-TMD of HIV-1 Env. (FIG. 2a) The TEM images of gold-polyphenol nanoparticles (AuNPs) and AuNP functionalized with NTA (NTA-AuNP). (FIG. 2b) ¹H NMR spectra the Foldon protein with C-terminal His6-tag in the absence and presence of Ni-NTA-AuNPs. Blue: 450 μl of 30 μM Foldon-His₆; Red: 450 μl of 30 μM Foldon-His₆ mixed with 100 μl of Ni-NTA functionalized Au-polyphenol nanoparticles (OD530=0.1) (the volume of the mixture were adjusted to 450 μl before NMR measurement). (FIG. 2c) Schematic illustration of unidirectional coating of bicelle-reconstituted MPER-TMD onto a functionalized AuNP. (FIG. 2d) The wide view of the negative staining EM (nsEM) image of Ni-NTA-AuNP-guided proteoliposome formation with the MPER-TMD. (FIG. 2e) nsEM images of the Ni-NTA-AuNP-supported MPERTMD proteoliposomes at two different magnifications. (FIG. 2f) nsEM images of DNA buckyball-supported MPER-TMD proteoliposomes at two different magnifications. (FIG. 2g) Analysis of FLAG-MPER-TMD-His6 orientation in Ni-NTA-AuNP-supported liposomes by antibody resin pull-down and SDS-PAGE. Lane 1: Ni-NTA-AuNP-supported MPER-TMD liposome; Lane 2: flow-through from anti-His6 resin after 30 minutes incubation; Lane 3: elution from anti-His6 resin; Lane 4: flow-through from anti-FLAG resin after 30 minutes incubation; Lane 5: elution from anti-FLAG resin.

FIG. 3A-3D. Unidirectional presentation of the MPER-TMD of HIV-1 Env in nanoparticle-supported liposomes. (FIG. 3a) Ni-NTA-AuNP supported MPER-TMD proteoliposomes treated by Anti-FLAG and Anti-His antibodies, respectively. (FIG. 3b) nsEM images (at two different magnifications) of the Ni-NTA-AuNP-supported proteoliposomes containing MPER-TMD with N-terminal FLAG tag and C-terminal His₆-tag incubated with anti-FLAG antibody. (FIG. 3c) nsEM image of a Ni-NTA-AuNP-supported proteoliposome containing MPER-TMD with N terminal FLAG tag and C-terminal His₆-tag incubated with anti-His₆ antibody at two different magnifications. (FIG. 3d) Analysis of FLAG-MPER-TMD-His₆ orientation in Ni-NTA-AuNP-supported liposome by antibody resin pull-down and SDS-PAGE. M: M.W. marker; Lane 1: Ni-NTA-AuNP-supported MPER-TMD liposome; Lane 2: flow-through from anti-His₆ resin after 30 minutes incubation; Lane 3: elution from anti-His₆ resin; Lane 4: flow-through from anti-FLAG resin after 30 minutes incubation; Lane 5: elution from anti-FLAG resin.

FIG. 4A-4B. Schematic illustration of AuNP synthesis and functionalization. (FIG. 4a) Reaction mechanism of gold nanoparticle synthesis and stabilization by tannic acid. Phenolic hydroxyl group (—OH) of tannic acid can reduce the gold precursor and coordinate with gold. The rest of polyphenol groups can act as surfactant to stabilize the gold ion with electrostatic interaction. (FIG. 4b) Glutaraldehyde is used to conjugate primary amine of lysine-NTA and nucleophilic carbons from tannic acid's phenol group.

FIG. 5A-5B. Characterization of polyphenol-stabilized gold nanoparticles. (FIG. 5a) Dynamic light scattering (DLS) of polyphenol-stabilized AuNPs. DLS result shows that AuNPs are very homogenous, with diameter around 47 nm. (FIG. 5b) Surface charges of AuNPs, NTA-AuNPs, and Ni-NTA-AuNPs were measured by Zetasizer Nano-ZS equipment. The zeta potentials (ζ) of AuNPs, NTA-AuNPs, and Ni-NTA-AuNPs are −51 mV, −36 mV, and −20.1 mV, respectively. The values are vastly different due to different surface properties of nanoparticles, indicating the successful functionalization of AuNPs at each stage.

FIG. 6A-6F. Controls for nanoparticle-supported liposome formation examined by negative stain EM. (FIG. 6a) Liposome formation from empty bicelles without Ni-NTA-AuNPs. 5 μl of bicelle buffer (45 mM DH6PC, 20 mM DMPC) was diluted by adding 400 μl of 25 mM phosphate buffer (pH 7.2). After DH6PC was removed through dialysis, 3.5 μl of sample was loaded onto copper grid. (FIG. 6b) Liposome formation from empty bicelles with Ni-NTA-AuNPs. 15 μl of Ni-NTA-AuNPs (OD530=0.1) was mixed with 5 μl of bicelle buffer (45 mM DH6PC, 20 mM DMPC). The mixture was diluted 20× by adding 400 μl of 25 mM phosphate buffer (pH 7.2). After DH6PC was removed, 3.5 μl of sample was loaded onto copper grid. (FIG. 6c) Liposome formation from bicelle-reconstituted MPER-TMD-His₆ with non-functionalized AuNPs. 15 μl of non-functionalized AuNP solution (OD530=0.1) was mixed with 5 μl of MPER-TMD sample (0.3 mM MPER-TMD, 45 mM DH6PC, 20 mM DMPC). The same dilution and dialysis steps were used before examined by negative staining EM. (FIG. 6d) Liposome formation from bicelle-reconstituted MPER-TMD-His₆ with Ni-NTA-functionalized AuNPs. The volume ratio between Ni-NTA-AuNP solution (OD530=0.1) and the MPER-TMD sample (0.3 mM, 45 mM DH6PC, 20 mM DMPC) is set at 1:1. Detailed liposome formation and negative staining EM procedures are described in methods. (FIG. 6e) Liposome formation from bicelle-reconstituted MPER-TMD-His₆ with Ni-NTA-functionalized AuNPs. The volume ratio between Ni-NTA-AuNP solution (OD530=0.1) and the MPER-TMD sample (0.3 mM, 45 mM DH6PC, 20 mM DMPC) is set at 3:1. (FIG. 6f) Liposome formation from bicelle-reconstituted MPER-TMD-His₆ with Ni-NTA-functionalized AuNPs. The volume ratio between Ni-NTA-AuNP solution (OD530=0.1) and the MPER-TMD sample (0.3 mM, 45 mM DH6PC, 20 mM DMPC) is set at 6:1. Note: Naked AuNPs and Ni-NTA-AuNPs could not be observed due to incompatibility with negative staining.

FIG. 7A-7B. Preparation of TriNTA-functionalized single stranded DNA. (FIG. 7a) The reaction scheme used to conjugate the primary amine of TriNTA to the thiol group of synthesized DNA via bifunctional PEG2-SMCC linker. In the scheme, the NHS ester of SMCC reacts with the amine group of TriNTA with extremely high efficiency (>95%). The SMCC maleimide group reacts with the thiol group of DNA. (FIG. 7b) TriNTA-functionalized DNA purification steps analyzed by agarose gel. The TriNTA-PEG2-SMCC, purified by HPLC, was mixed with the thiol-modified L strand to form TriNTA-L conjugate. After the reaction, the TriNTA-L was charged with Ni′. Detailed procedure can be found in methods section. The gel lanes are: L—DNA ladder; 1—TriNTA-L reaction mixture; 2—flow-through of the reacted solution from Foldon-His₆-tag resin; 3—wash of the resin to remove unbound species; 4—elution from the resin with 0.2 M imidazole; 5—further elution with 0.5 M imidazole.

FIG. 8A-8B. DNA buckyball formation. (FIG. 8a) Agarose gel analysis of DNA buckyball formation using the TriNTA-functionalized L strand and regular M and S strands. Lane 1: pure Ni-TriNTA-L strand; Lane 2: pure M strand; Lane 3: pure S strand; Lane 4: after direct mixing of Ni-TriNTA-L, M, and S, at 1:3:3 molar ratio; Lane 5: after two-step annealing treatment of the mixed sample (details in Methods). Comparison of the higher and lower bands suggests that the efficiency of DNA buckyball formation is ˜90%. (FIG. 8b) The assembled DNA buckyball sample was negatively stained and observed using the CM10 electron microscope. The EM image shows mostly homogenous assemblies with diameter ˜80 nm.

FIG. 9A-9C. Proteoliposome formation guided by functionalized DNA buckyballs (FIG. 9a) Negative stain EM image of liposomes formed by mixing functionalized DNA buckyballs and empty bicelles, showing highly inhomogeneous liposomes. 143 μl of DNA buckyball ([Tri-NTA-L]=70 nM) was mixed with 1 μl of bicelle buffer (45 mM DH6PC, 20 mM DMPC). The mixture was treated with PD-10 column twice, then examined by negative staining EM. Detailed procedures are described in methods. (FIG. 9b) Negative stain EM image of liposomes formed by mixing functionalized DNA buckyballs and bicelle-reconstituted MPERTMD-His₆, showing much better defined shape and size. 143 μl of DNA buckyball ([Tri-NTA-L]=70 nM) was mixed with 1 μl of MPER-TMD-His₆ (0.3 mM MPER-TMD, 45 mM DH6PC, 20 mM DMPC). Note: some incompletely assembled buckyballs could also support liposome assembly, resulting in smaller liposomes. (FIG. 9c) Negative stain EM image of the naked DNA buckyball, showing that the size is consistent with those observed in (FIG. 9b).

FIG. 10A-10C. Unidirectional presentation of GP160 in nanoparticle supported liposomes. (FIG. 10a) illustration of expression, purification, and coating of GP160 in nanoparticle supported liposome. (FIG. 10b) SDS-PAGE of purification of gp160. (FIG. 10c) TEM images of coating gp160 to the surface of functionalized nanoparticle.

FIG. 11A-11C. Immunogenicity of Ni-NTA-AuNP-supported MPER-TMD proteoliposomes. (FIG. 11a) Immunization schedule of 5 guinea pigs. Serum samples were collected at weeks 4 and 12. (FIG. 11b) ELISA analysis of serum for anti-MPER-TMD antibodies. ELISA plates were coated with MPER-TMD in regular liposomes. Sera were added in serial dilutions (2700, 900, 300, 100) and detected with a horseradish peroxidase (HRP)-conjugated rabbit anti-guinea pig secondary antibody for total IgG ELISAs. (FIG. 11c) ELISA plate incubated with empty liposomes.

FIG. 12A-12I. The ELISA results show that all of the sera after the first immunization with AuNP-supported proteoliposomes already contained anti-MPER-TMD antibodies (FIG. 12A; squares), and that sera from the second and third immunization elicited antibodies that bind much stronger and more robust to MPER-TMD (FIG. 12B and FIG. 12C; squares). For conventional proteoliposomes, the sera from first immunization showed almost no specific ELISA signal against MPER-TMD (FIG. 12A; circles). Although sera from the second and the third immunization induced more MPER-TMD specific antibodies (FIG. 12B and FIG. 12C; circles), their ELISA signal intensity are only 30-50% of those from the corresponding immunization using the AuNP-supported proteoliposomes. As a negative control, no strong binding to the empty liposomes was observed (FIG. 12D, FIG. 12E, and FIG. 12F). The ELISA signals averaged over 4 mice in each group show that anti-MPER-TMD antibody production induced by the AuNP-supported proteoliposomes is at least twofold stronger than that induced by conventional proteoliposomes for all dilution factors (FIG. 12G, FIG. 12H, and FIG. 12I).

Described herein is an assembly including a substrate, a coating, a quantity of lipids including a protein with a tag, wherein the coating is attached to the substrate and bound to the tag. In various embodiments, the substrate is globular, solid, hollow, porous, multi-layer, and combinations thereof. In various embodiments, the substrate is spherical, cubic, tetrahedral, tubular, or in any three dimensional shape. In various embodiments, the substrate is a nanoparticle. In various embodiments, the nanoparticle includes a gold nanoparticle, silver nanoparticle, platinum nanoparticle, silicon dioxide nanoparticle, porous silicon nanoparticle, polymer nanoparticle, and/or complex nanoparticle. In various embodiments, the substrate is a biological molecule. In various embodiments, the biological molecule is selected from the group consisting of: DNA, RNA, PNA, LNA, and protein. In various embodiments, the DNA is a buckyball, cube, tetrahedron, dodecahedron, pyramid, tube, or stick. In various embodiments, the DNA includes a di-sulfide modifier, amino modifier, azide modifier, acrydite modifier, alkyne modifier, biotin, and/or digoxigenin. In various embodiments, the coating includes polyphenol, tannic acid, catechin, dopamine, theaflavin, anthocyanidin, and derivatives thereof. In various embodiments, the coating includes one or more molecules selected from the group consisting of: PEG-SMCC, AMAS, BMPS, GMBS, MBS, EMCS, SMPB, SMPH, SPDP, and SMPT. In various embodiments, the coating includes lysine, and/or cysteine. In various embodiments, the coating includes NTA-Ni, antibodies, nanobodies, biotin, and/or streptavidin. In various embodiments, the lipids are bicelles, synthetic lipids, extracts from host, and combinations thereof. In various embodiments, the tag is one or more tags selected from the group consisting of: histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag. In various embodiments, the coating attached to the substrate and bound to the tag externally presents a feature of the protein.

In other embodiments, the assembly is injected into an animal. In various embodiments, the animal is a mouse, rabbit, goat, horse or other animal used to general antibodies. In various embodiments, the injection supports generation of antibodies against the protein. In various embodiments, the injection supports generation of antibodies of greater potency and/or specificity to the protein, when compared to injection with a conventional protein presentation (i.e., liposome). In various embodiments, the potency is about 10-20%, 20-30%, 30-40% 40-50%, 50-60%, 60% or more when injecting the assembly compared to the conventionally generated protein presentation. In various embodiments, the injection supports generation of antibodies that do not generate an immunogenic response in comparison to conventional protein presentation (i.e., liposome).

Described herein is a quantity of antibodies made by the aforementioned methods. In various embodiments, the quantity of antibodies is made by injecting an assembly into an animal, wherein the assembly comprises a substrate, a coating, a quantity of lipids including a protein with a tag, wherein the coating is attached to the substrate and bound to the tag. In various embodiments, the antibodies are specific against a protein, or antigenic fragment thereof, the protein, or antigenic fragment thereof otherwise incapable of antibody generation in the absence of unidirectional presentation. In various embodiments, the antibodies are of greater potency and/or specificity to the protein, when compared to injection with a conventional protein presentation (i.e., liposome). In various embodiments, the potency is about 10-20%, 20-30%, 30-40% 40-50%, 50-60%, 60% or more when injecting the assembly compared to the conventionally generated protein presentation.

Also described herein is a method, including attaching a coating to a substrate, and adding a functional moiety to the coating. In various embodiments, the substrate is globular, solid, hollow, porous, multi-layer, and combinations thereof. In various embodiments, the substrate is spherical, cubic, tetrahedral, tubular, or in any three dimensional shape. In various embodiments, the substrate is a nanoparticle. In various embodiments, the nanoparticle includes a gold nanoparticle, silver nanoparticle, platinum nanoparticle, silicon dioxide nanoparticle, porous silicon nanoparticle, polymer nanoparticle, and/or complex nanoparticle. In various embodiments, the substrate is a biological molecule. In various embodiments, the biological molecule is selected from the group consisting of: DNA, RNA, PNA, LNA, and protein. In various embodiments, the DNA is a buckyball, cube, tetrahedron, dodecahedron, pyramid, tube, or stick. In various embodiments, the DNA includes a di-sulfide modifier, amino modifier, azide modifier, acrydite modifier, alkyne modifier, biotin, and/or digoxigenin. In various embodiments, the coating includes polyphenol, tannic acid, catechin, dopamine, theaflavin, anthocyanidin, and derivatives thereof. In various embodiments, the coating includes PEG-SMCC AMAS, BMPS, GMBS, MBS, EMCS, SMPB, SMPH, SPDP, and SMPT. In various embodiments, the coating includes lysine and/or cysteine. In various embodiments, the functional moiety includes NTA-Ni, antibodies, nanobodies, biotin, and/or streptavidin. In various embodiments, the tag is one or more tags selected from the group consisting of: histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag. In various embodiments, the method includes adding a bicelle including a protein with a tag to the substrate. In various embodiments, the method includes a bicelle attached to a detergent that is removed prior to addition. In various embodiments, attachment of the tag is to the functional moiety, externally presents feature of the protein.

Further described herein is a method of detecting a protein, including incubating a tagged protein with a lipid and a detergent to form an assembly, removing the detergent, incubating the assembly in the presence of a coated substrate, wherein the coated substrate includes a functional moiety capable of binding to the tagged protein, forming proteoliposomes on the coated substrate to externally present a feature of the tagged protein, and detecting the protein using the externally presented feature. In various embodiments, the protein is a membrane bound protein, including transmembrane protein, membrane transport protein, channel protein, membrane receptor, membrane anchored protein, and membrane protein complex.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22_{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul. 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

The Inventors sought to use nanoparticles as substrates to guide proteoliposome assembly. By modifying nanoparticles with functional moieties that specifically recruit affinity-tagged membrane proteins in bicelles, the Inventors could form proteoliposome around the nanoparticle where the proteins are presented in a unidirectional manner. The Inventors have demonstrated this approach, named Supported ProteoLiposome for Antigen Directed Display (SPLAnDiD), for a membrane fragment of the HIV-1 envelope glycoprotein (Env) encompassing the transmembrane (TM) domain and the membrane-proximal external region (MPER).

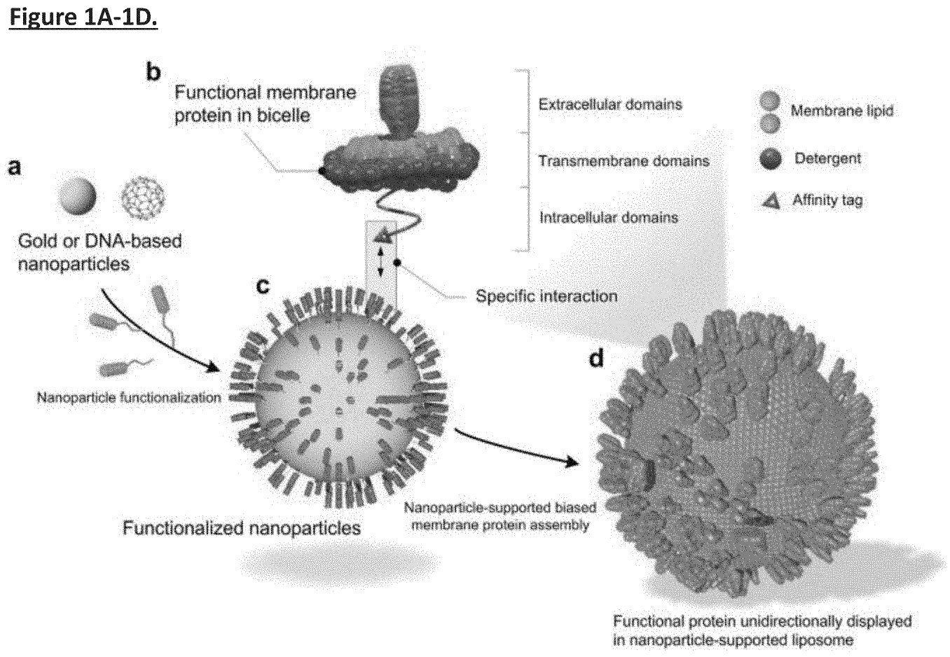

The design concept, illustrated in FIG. 1a-d, is to specifically recruit membrane proteins solubilized in lipid/detergent bicelles (that closely mimic a lipid bilayer) to the surface of a globular shaped nanoparticle, also referred to as the substrate. As detergent is removed, the protein-containing bicelles will grow on the substrate surface to form proteoliposomes. Nanoparticles can take on various forms and compositions. For biological or medical applications, it is important that the particles are biocompatible with no major toxic or immunogenic risk to biological organisms. The Inventors experimented with two different types of nanoparticles (FIG. 1a). One is polyphenol-stabilized gold nanoparticle (AuNP), which is solid and can be fabricated to various sizes ranging from 3 to 50 nm by exploiting the coordination and stabilization of gold ions with polyphenol moieties. AuNPs are biocompatible and have been approved by FDA as Generally Recognized as Safe (GRAS) and for drug use. Polyphenol moieties can be further functionalized via their hydroxyl groups and active carbon sites. Another form of nanoparticle is the hollow DNA buckyball assembled by DNA origami. DNAs are generally non-immunogenic. A variety of modifications can be incorporated into DNA during synthesis for functionalization.

Described herein is an assembly including a substrate, a coating, a quantity of lipids including a protein with a tag, wherein the coating is attached to the substrate and bound to the tag. In various embodiments, the substrate is globular, solid, hollow, porous, multi-layer, and combinations thereof. In various embodiments, the substrate is globular. In various embodiments, the substrate is spherical, cubic, tetrahedral, tubular, or in any three dimensional shape. One of ordinary skill understands the substantially three dimensional shapes supramolecule DNA is capable of forming. In various embodiments, the substrate is spherical. In various embodiments, the substrate is a nanoparticle. In various embodiments, the nanoparticle includes a gold nanoparticle, silver nanoparticle, platinum nanoparticle, silicon dioxide nanoparticle, porous silicon nanoparticle, polymer nanoparticle, and/or complex nanoparticle. In various embodiments, the nanoparticle is a gold nanoparticle. In various embodiments, the substrate is a biological molecule. In various embodiments, the biological molecule is selected from the group consisting of: DNA, RNA, PNA, LNA, and protein. In various embodiments, the biological molecule is DNA. In various embodiments, the DNA is a buckyball, cube, tetrahedron, dodecahedron, pyramid, tube, or stick. In various embodiments, the DNA is a buckyball. In various embodiments, the DNA includes a di-sulfide modifier, amino modifier, azide modifier, acrydite modifier, alkyne modifier, biotin, and/or digoxigenin. In various embodiments, the DNA includes a di-sulfide modifier. In various embodiments, the coating includes polyphenol, tannic acid, catechin, dopamine, theaflavin, anthocyanidin, and derivatives thereof. In various embodiments, the coating includes polyphenol. In various embodiments, the coating includes one or more molecules selected from the group consisting of: PEG-SMCC, AMAS, BMPS, GMBS, MBS, EMCS, SMPB, SMPH, SPDP, and SMPT. In various embodiments, the coating includes PEG-SMCC. In various embodiments, the coating includes lysine, and/or cysteine. In various embodiments, the coating includes lysine. In various embodiments, the coating includes NTA-Ni, antibodies, nanobodies, biotin, and/or streptavidin. In various embodiments, the coating includes NTA-Ni. In various embodiments, the lipids are bicelles, synthetic lipids, extracts from host, and combinations thereof. In various embodiments, the lipids are bicelles. In various embodiments, the tag is one or more tags selected from the group consisting of: histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag. In various embodiments, the tag is histidine. In various embodiments, the coating attached to the substrate and bound to the tag externally presents a feature of the protein.

For example, an assembly including a substrate, a coating, a quantity of lipids including a protein with a tag, wherein the coating is attached to the substrate and bound to the tag can include a substrate that is globular or spherical, a substrate that is a nanoparticle including a gold nanoparticle or DNA in the form of a buckyball. The DNA can include a di-sulfide modifier. In various embodiments, the coating includes polyphenol. In various embodiments, the coating includes PEG-SMCC. In various embodiments, the coating includes lysine. In various embodiments, the coating includes NTA-Ni. In various embodiments, the lipids are bicelles. In various embodiments, the tag is histidine. In various embodiments, the coating attached to the substrate and bound to the tag externally presents a feature of the protein. In all instances, a feature of the protein is a feature of interest, particularly spatially relevant features of a protein with a particular orientation in biological systems when bound to a membrane. This includes, for example, membrane bound proteins, such as transmembrane protein, membrane transport protein, channel protein, membrane receptor, membrane anchored protein, and membrane protein complex.

Also described herein is a method, including attaching a coating to a substrate, and adding a functional moiety to the coating. In various embodiments, the substrate is globular, solid, hollow, porous, multi-layer, and combinations thereof. In various embodiments, the substrate is spherical, cubic, tetrahedral, tubular, or in any three dimensional shape. In various embodiments, the substrate is a nanoparticle. In various embodiments, the nanoparticle includes a gold nanoparticle, silver nanoparticle, platinum nanoparticle, silicon dioxide nanoparticle, porous silicon nanoparticle, polymer nanoparticle, and/or complex nanoparticle. In various embodiments, the substrate is a biological molecule. In various embodiments, the biological molecule is selected from the group consisting of: DNA, RNA, PNA, LNA, and protein. In various embodiments, the DNA is a buckyball, cube, tetrahedron, dodecahedron, pyramid, tube, or stick. In various embodiments, the DNA includes a di-sulfide modifier, amino modifier, azide modifier, acrydite modifier, alkyne modifier, biotin, and/or digoxigenin. In various embodiments, the coating includes polyphenol, tannic acid, catechin, dopamine, theaflavin, anthocyanidin, and derivatives thereof. In various embodiments, the coating includes PEG-SMCC AMAS, BMPS, GMBS, MBS, EMCS, SMPB, SMPH, SPDP, and SMPT. In various embodiments, the coating includes lysine and/or cysteine. In various embodiments, the functional moiety includes NTA-Ni, antibodies, nanobodies, biotin, and/or streptavidin. In various embodiments, the tag is one or more tags selected from the group consisting of: histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag. In various embodiments, the method includes adding a bicelle including a protein with a tag to the substrate. In various embodiments, the method includes a bicelle attached to a detergent that is removed prior to addition. In various embodiments, attachment of the tag is to the functional moiety, externally presents feature of the protein.

For example, the method includes using an assembly including a substrate, a coating, a quantity of lipids including a protein with a tag, wherein the coating is attached to the substrate and bound to the tag can include a substrate that is globular or spherical, a substrate that is a nanoparticle including a gold nanoparticle or DNA in the form of a buckyball. The DNA can include a di-sulfide modifier. In various embodiments, the coating includes polyphenol. In various embodiments, the coating includes PEG-SMCC. In various embodiments, the coating includes lysine. In various embodiments, the coating includes NTA-Ni. In various embodiments, the lipids are bicelles. In various embodiments, the tag is histidine. In various embodiments, the coating attached to the substrate and bound to the tag externally presents a feature of the protein. In various embodiments, the method includes altering the assembly components in different molar ratios, for example, 1:1, 1:2, 1:3, 1:4, 1:6, 1:7. 1:8, 1:9, 1:10, 1:10-50, 1:50-100 and all variable ranges in-between.

Further described herein is a method of detecting a protein, including incubating a tagged protein with a lipid and a detergent to form an assembly, removing the detergent, incubating the assembly in the presence of a coated substrate, wherein the coated substrate includes a functional moiety capable of binding to the tagged protein, forming proteoliposomes on the coated substrate to externally present a feature of the tagged protein, and detecting the protein using the externally presented feature. In various embodiments, the protein is a membrane bound protein, including transmembrane protein, membrane transport protein, channel protein, membrane receptor, membrane anchored protein, and membrane protein complex. In various embodiments, the protein is a membrane bound protein.

### Example 1

### Nanoparticle Preparations

Gold-polyphenol nanoparticle production and functionalization. 0.8 mg/ml of tannic acid (Sigma Aldrich) was prepared in ddH2O. Chloroauric acid (Sigma Aldrich) was added to tannic acid solution drop by drop to the final concentration of 0.4 mM. The mixture was incubated at room temperature with stirring at 800 rpm for 20 minutes to form polyphenol-stabilized gold nanoparticles (AuNPs). AuNPs were spun down at 12,000 g for 10 minutes. Pellet was then washed with ddH2O. Resuspended AuNPs in ddH2O was thoroughly sonicated before centrifugation again. The centrifugation and washing steps were repeated twice. AuNPs were then mixed with 0.04% glutaraldehyde (Electron Microscopy Sciences) and 0.5 mg/ml Nα,Nα-Bis(carboxymethyl)-L-lysine (Lysine-NTA) (Sigma Aldrich) at 45° C. for 1 hour. The conjugated NTA-AuNPs were then spun down and washed with ddH2O for three times. 0.5 mM NiCl2 was added to the NTA-AuNP solution. Ni-NTA-AuNPs were then washed with ddH2O for four times and stored at 4° C. upon further use.

### Example 2

### Zeta-Potential Measurement

The zeta-potentials were measured using a Zetasizer Nano-ZS (Malvern Instruments, UK) with a 633 nm He—Ne ion laser. The capsules were suspended in 10 mM phosphate buffer (pH 7.4) before adding different nanoparticle solutions. Measurements were repeated three times. The results were expressed as the mean and standard deviation obtained from the three measurements.

Interaction between Ni-NTA-AuNPs and His6-tag. Foldon is the C-terminal domain of T4 fibritin containing 27 residues and forms highly-stable trimer. Foldon with C-terminal His6-tag was cloned into the pET-15 vector and expressed in BL21(DE3) cells at 37° C. (induced with 1 mM isopropyl-β-d-thiogalactopyranoside (IPTG) for 6 hours). The protein was purified by Ni-NTA affinity (HisPur Ni-NTA resin, Thermo Fisher) and size exclusion chromatography (superdex 75 column, GE Healthcare). The NMR oneone echo experiment was used to record the 1D 1H spectrum of a 450 μl Foldon sample (30 μM Foldon, 25 mM phosphate, 50 mM NaCl, pH 7.2) before and after mixing with 100 μl of Ni-NTA-AuNP solution (OD530=0.1) in the same buffer. In the latter, the volume of the mixture was concentrated back to 450 μl before NMR measurement.

### Example 3

### MPER-TMD Plasmid Construction, Expression and Purification

The MPER-TMD corresponds to a fragment of HIV-1 gp41 (clade D, isolate 92UG024.2) spanning residues 660-710; it contains the entire MPER (residues 660-683) and the TMD (residues 684-705). FLAG-tag and His6-tag sequences were added to the N- and C-termini of the MPER-TMD, respectively. The FLAG-MPER-TMD-His6 DNA was cloned into the pMM-LR6 vector as a fusion to the C-terminus of the trpLE sequence.

The MPER-TMD plasmid was transformed into E. coli BL21(DE3) for expression. Cell cultures were grown at 37° C. in LB media until OD600 reached 0.6, and cooled to 22° C. before induction with 100 μM isopropyl β-D-thiogalatopyranoside (IPTG) at 22° C. for overnight. The MPER-TMD was extracted from inclusion bodies, cleaved by cyanogen bromide, and purified by HPLC as described previously. The purified MPER-TMD were lyophilized and validated by SDS-PAGE and MALDI-TOF mass spectrometry.

### Example 4

### Reconstitution of MPER-TMD in Bicelles

2 mg of lyophilized MPER-TMD powder was mixed with 9 mg of 1,2-dimyristoylsn-Glycero-3-Phosphocholine (DMPC, Avanti Polar Lipids) in hexafluoro-isopropanol. The mixture was blown dry to a thin film in a glass vial under nitrogen gas, followed by overnight lyophilization. The dried thin film was dissolved in 3 ml of 8 M urea containing 20 mg of 1,2-dihexanoyl-sn-Glycero-3-Phosphocholine (DH6PC, Avanti Polar Lipids) and 4 mg of 1,2-diheptanoylsn-Glycero-3-Phosphocholine (DH7PC, Avanti Polar Lipids). The mixture was dialyzed twice against phosphate buffer (pH 7.2, 50 mM NaCl) (1 L each time) to remove urea. Additional DH6PC was added to the sample every hour to compensate its loss due to dialysis. The DMPC:DH6PC ratio was controlled between 0.5 and 0.6 by 1D NMR. Bicelle reconstituted MPER-TMD was concentrated to ˜1 ml (around 0.3 mM) after dialysis.

### Example 5

### Ni-NTA-AuNP Supported Proteoliposome Formation

The Ni-NTA-AuNP solution (OD530=0.1) and the bicelles reconstituted MPER-TMD solution (˜0.3 mM) were mixed at the ratio of 3:1 (vol/vol). The mixture was diluted 20 times by adding phosphate buffer (pH 7.2, 50 mM NaCl) before dialyzed against the same phosphate buffer to remove DH6PC detergent. 10 kDa cut off dialysis cassette was used (Life technology). Buffer was changed every 3 hours for at least 6 times at 4° C.

### Example 6

### Imaging Interaction Between Antibody and MPER-TMD Proteoliposome

Anti-FLAG or anti-His₆ antibody (Sigma) was added to Ni-NTA-AuNP-supported proteoliposome containing the FLAG-MPER-TMD-His₆. The amount of antibody was added at 1:1 molar ratio of antibody:MPER-TMD. The concentration of the MPER-TMD was estimated based on the assumption that all applied MPER-TMD (with known amount) were incorporated into the AuNP-supported proteoliposome. Antibodies and nanoparticle were mixed for 10 minutes at room temperature before analysis by negative staining EM.

### Example 7

### DNA Buckyball Formation, Supported Proteoliposome Formation

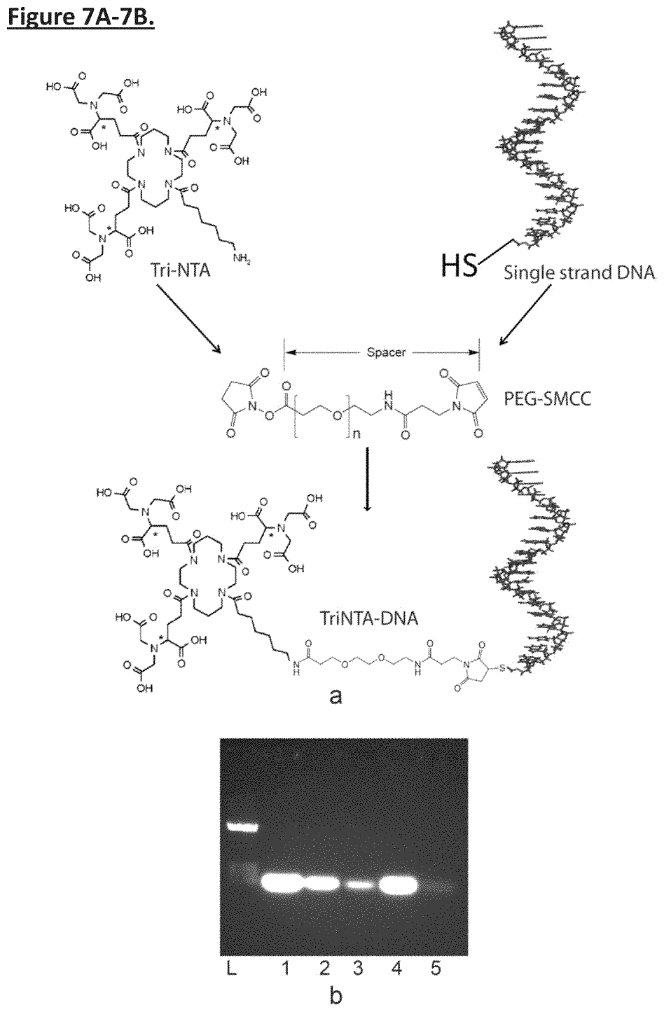

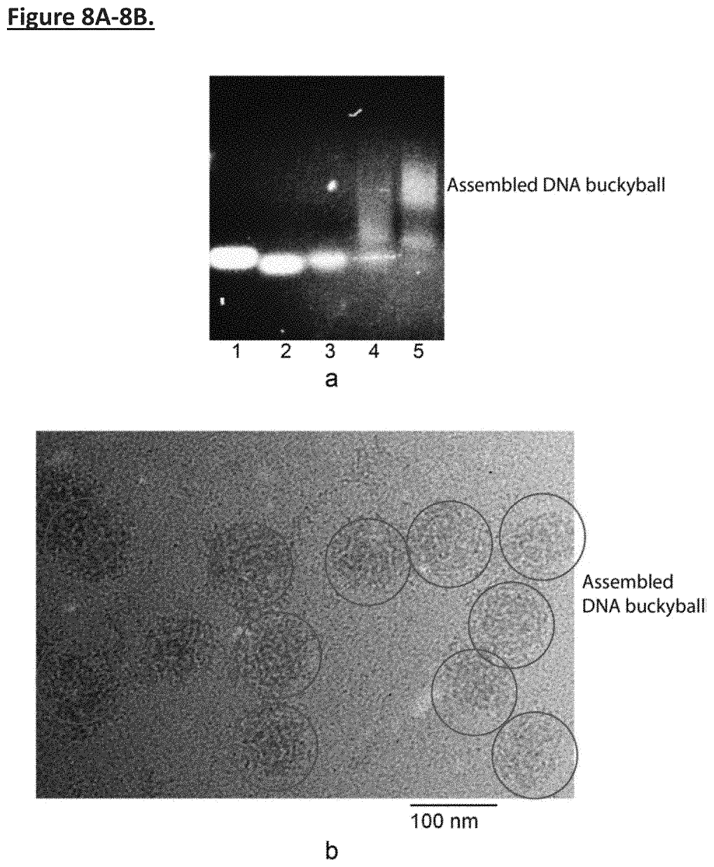

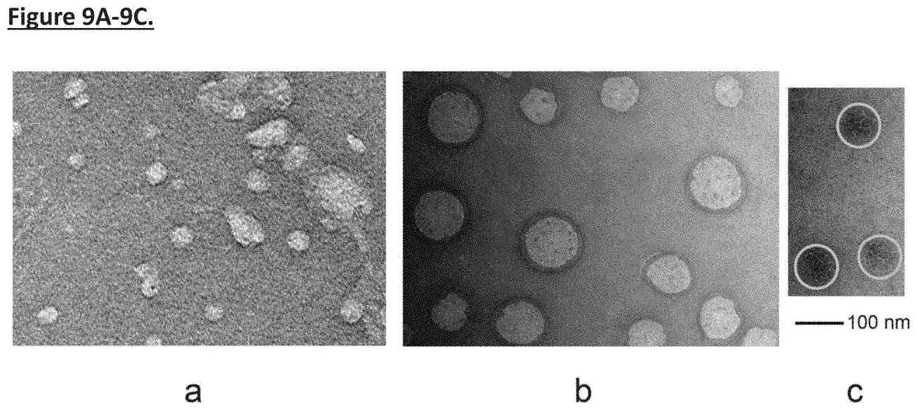

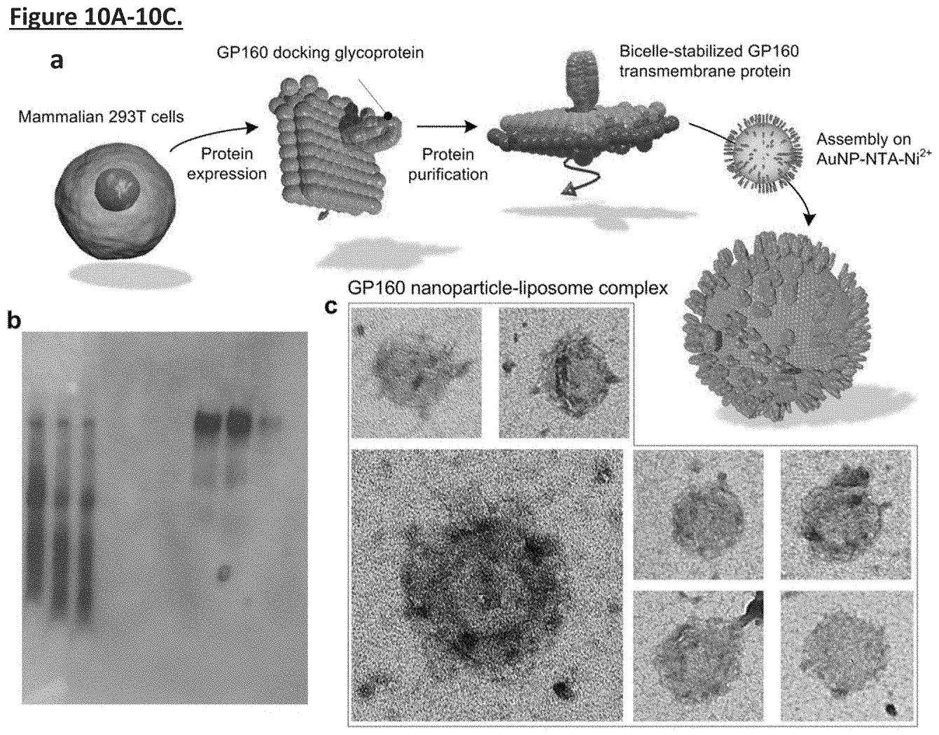

DNA sequences for buckyball assembly were adapted from previous work; long strand (L):aggcaccatcgtaggtttcttgccaggcaccatcgtaggtttcttgccagg-caccatcgtaggtttcttgcc [SEQ ID NO: 1]; medium stand (M): tagcaacctgcctggcaagcctacgatggacacggtaacgcc [SEQ ID NO: 2]; short strand (S): ttaccgtgtggttgctaggcg [SEQ ID NO: 3]. Thiol modifier C6 S—S was added to the 3′ of the long strand. TriNTA with a free primary amine (FIG. 7a) was synthesized by Medicilon Inc. (Shanghai, China) and stored as dry powder at −20° C. The bi-functional crosslinker PEG2-SMCC (succinimidyl 4-(N-maleimidomethyl)cyclohexane-1-carboxylate) was incubated with TriNTA (with 4:1 molar ratio) in 100 mM sodium carbonate buffer (pH 8.0) for 1 hour, which achieved >95% reaction efficiency. The SMCC-linked TriNTA was then purified by RP-HPLC using a Zorbax SB-C18 semi-preparative column (Agilent) and a gradient of 5-20% acetonitrile in 0.1 M Triethylammonium Acetate (TEAA) (Calbiochem). SMCC-TriNTA was lyophilized and stored at −80° C. upon further use. To functionalize the L DNA strand with TriNTA, 100 μM of thiol-modified L strand was dissolved in 25 mM HEPES buffer (pH 7.4), and reduced by 20 mM DTT for 10 minutes. The DTT-treated L solution was applied to PD-10 column (GE Healthcare) to remove DTT, followed by immediate mixing with 2 times excess amount of SMCC-TriNTA in HEPES buffer (pH 7.4). The reaction mixture was degassed for 30 min and incubated for additional 30 min at room temperature. Ni2+ was added to TriNTA-L conjugate at 3:1 molar ratio. The Ni-TriNTA-L conjugate was purified using a His6-tag column, made in the lab by chemically linking the Foldon-His6-tag to NHS-activated agarose resin (Thermo Fisher Scientific). The Ni-TriNTA-L conjugate was mixed with the Foldon-His₆-tag resin for 30 minutes, and eluted with 200 mM imidazole (FIG. 7b). Pure Ni-TriNTA-L conjugate was stored in TA/Mg2+ buffer (40 mM Tris, 20 mM acetic acid, 12.5 mM magnesium acetate, pH 8.0) after removing imidazole. Finally, the Ni-TriNTA-L, the M, and the S were mixed at molar ratio of 1:3:3 in TA/Mg²⁺ buffer. The final concentration of TriNTA-L was adjusted to 70 nM. The DNA buckyball assembly was achieved using two steps of annealing: 1) from 95° C. to 65° C., at a rate of 1 degree per 15 min; 2) from 65° C. to 20° C., at a rate of 1 degree per hour. DNA buckyball assembly was accessed by electrophoresis using 0.5% agarose gel (FIG. 8a). The DNA buckyball solution was concentrated ([TriNTA-L]≥400 nM) before examination by negative staining EM.

Bicelle-reconstituted MPER-TMD (with C-terminal His6-tag) was mixed with the TriNTA functionalized DNA buckyball at the molar ratio of 1 trimeric MPER-TMD to 1 TriNTA-L strand. The mixture was passed through PD-10 column twice to remove DH6PC detergent. The elution from PD-10 was concentrated ([TriNTA-L]≥400 nM) before analysis by negative staining EM.

### Example 8

### Electron Microscopy Sample Preparation

To examine AuNP alone by EM, 2.5 ul of AuNP solution (OD530=0.1) was loaded onto nickel grid with formvar/carbon film (Electron Microscopy Sciences), and dried in air for at least 2 hours. For DNA buckyball and all nanoparticle-supported liposomes, samples were loaded onto copper grid with formvar/carbon film (Electron Microscopy Sciences). The Ni-NTA-AuNP-supported MPER-TMD proteoliposomes were negatively stained by 1.5% Uranyl formate. DNA buckyball and DNA buckyball-supported MPER-TMD proteoliposomes were first concentrated ([TriNTA-L]≥400 nM), and then negatively stained by 1.5% Uranyl formate. EM images were taken using the CM10 electron microscope (Philips).

### Example 9

### Results

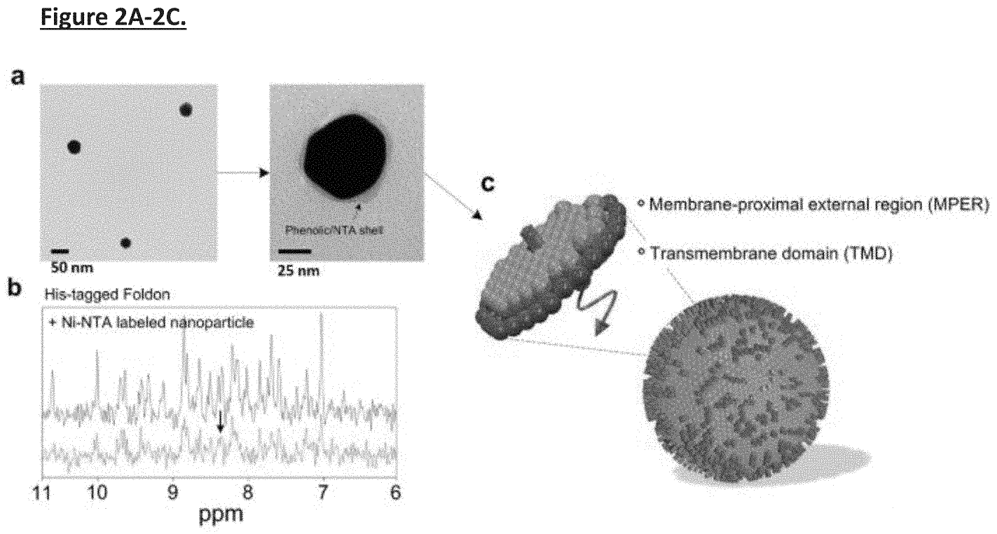

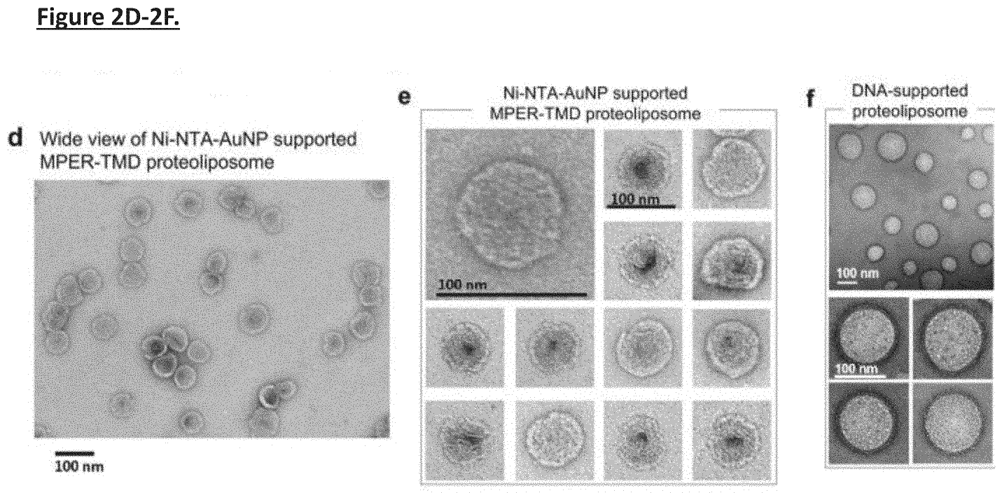

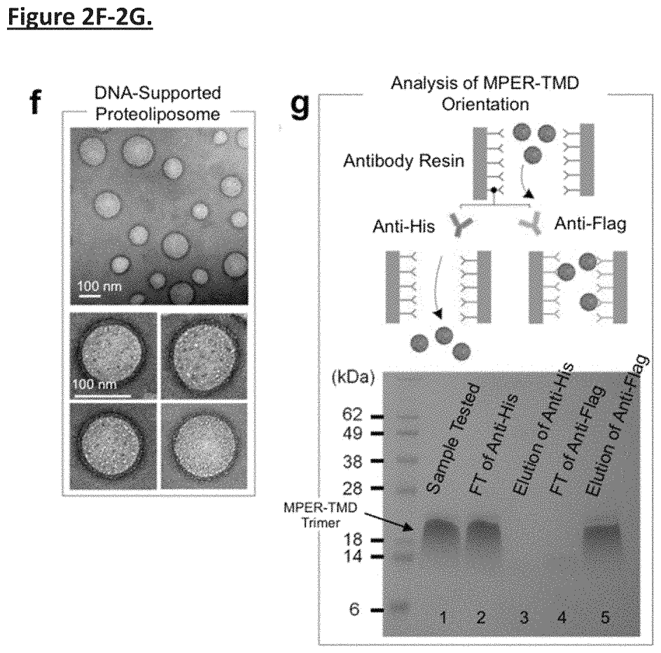

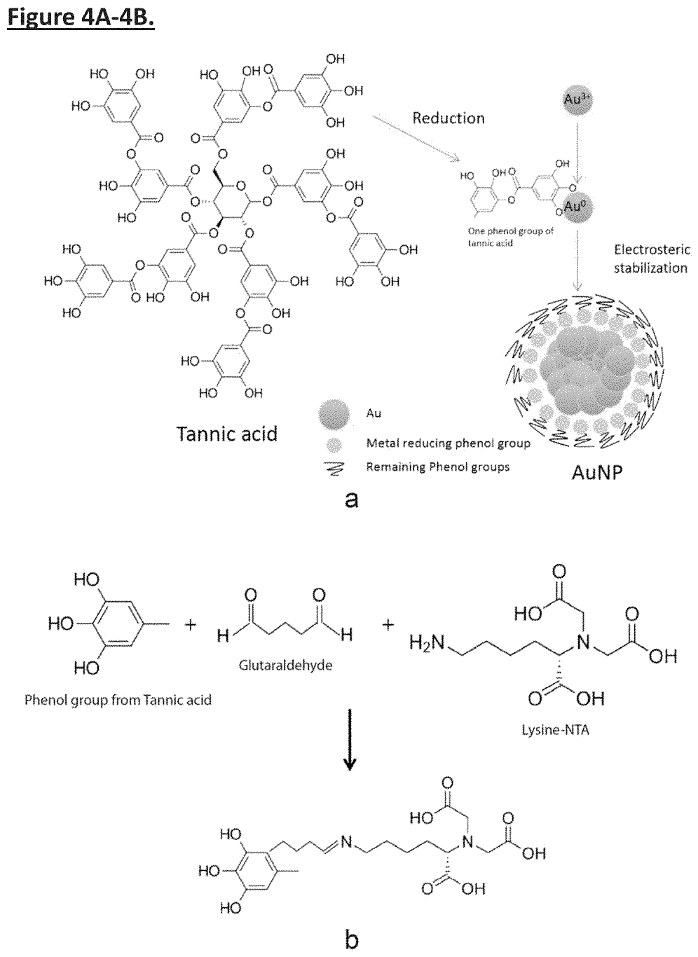

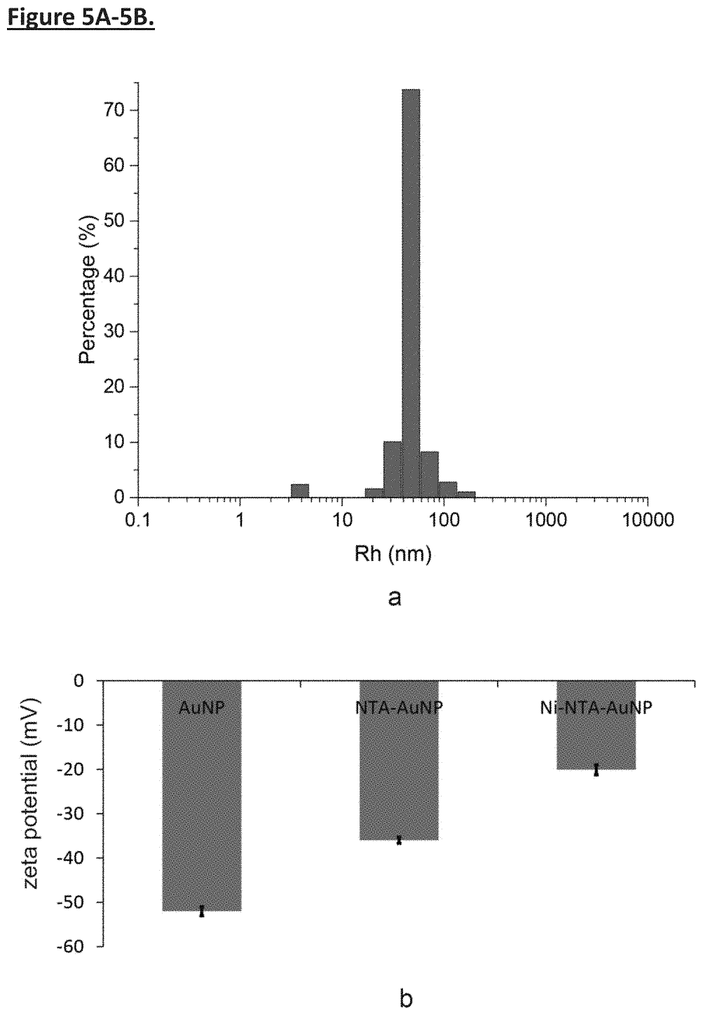

In the Inventors' assembly method, membrane proteins are purified and solubilized in bicelles composed of DMPC and DH6PC (Experimental Procedures) (FIG. 1b). An affinity tag is placed at the cytoplasmic end of the protein for directing the protein to the nanoparticles. In the current study, this affinity tag is a polyhistidine tag (His₆-tag), and the functional moieties on the nanoparticles are Ni-NTAs (FIG. 1c). Only bicelles containing the membrane protein of interest will specifically “glue” to the nanoparticle surface via (Ni-NTA)-(His₆-tag) interaction, and since the His₆-tag is on the cytoplasmic side of the protein, the protein-bicelle complex will be “glued” in a unidirectional manner. As DH6PC is removed, the bicelles will merge and form proteoliposome encapsulating the nanoparticle (FIG. 1d). AuNPs were synthesized by mixing the gold precursor, chloroauric acid (HAuCl₄), and tannic acid (TA) solution as described previously. Phenolic hydroxyl group (—OH) of TA attaches to the surface of the gold along with the rest of polyphenol to act as surfactant to stabilize the metal ion with electrostatic interaction (FIG. 4a). The size of the AuNPs can be precisely controlled by the ratio of chloroauric acid to TA and their mixing speed. The Inventors first prepared AuNPs with average diameter of ˜47 nm, as shown by transmission electron microscopy (TEM) (FIG. 2a) and dynamic light scattering (DLS)(FIG. 5a). The AuNPs were then functionalized with lysine-NTA (Experimental Procedures) by conjugating the amine groups of lysine-NTA and the nucleophilic carbons of the polyphenol groups via glutaraldehyde (FIG. 2a, 4b). The NTA-AuNPs were charged with Ni2+. The surface charges of AuNPs, evaluated as the zeta potential (ξ), were monitored during the functionalization process. As shown in FIG. 5b, the polyphenol-stabilized AuNPs are negatively charged (ξ=−52 mV) due to deprotonation of the hydroxyl groups. The ξ increased to −36 mV after conjugation with lysine-NTA and increased further to −20.1 mV after addition of Ni²⁺, as a result of modification of polyphenol groups and coordination with positively charged Ni²⁺, respectively. Finally, to examine the ability of the Ni-NTA functionalized AuNPs (Ni-NTA-AuNPs) to specifically interact with His₆-tag, the Inventors recorded 1D ¹H NMR spectrum of the Foldon protein containing C terminal His₆-tag (Foldon-His₆) before and after mixing with the Ni-NTA-AuNPs (Methods). The Foldon NMR signals were essentially ablated upon the addition of Ni-NTA-AuNPs (FIG. 2b), indicating strong binding of Foldon-His₆ to the nanoparticles that caused NMR signals to decay rapidly. Having generated Ni-NTA-AuNPs, the Inventors next tested the above scheme of proteoliposome formation using a fragment of HIV-1 Env that contains the membrane-proximal external region (MPER) and the transmembrane domain (TMD). This fragment (residues 660-710) is derived from a clade D HIV-1 isolate 92UG024.2 (designated MPER-TMD). The MPER is one of the most conserved regions of HIV-1 Env and bears epitopes of broadly neutralizing antibodies from infected individuals. Therefore, methods for presenting the MPER to human immune system are of strong interest to HIV vaccine development.

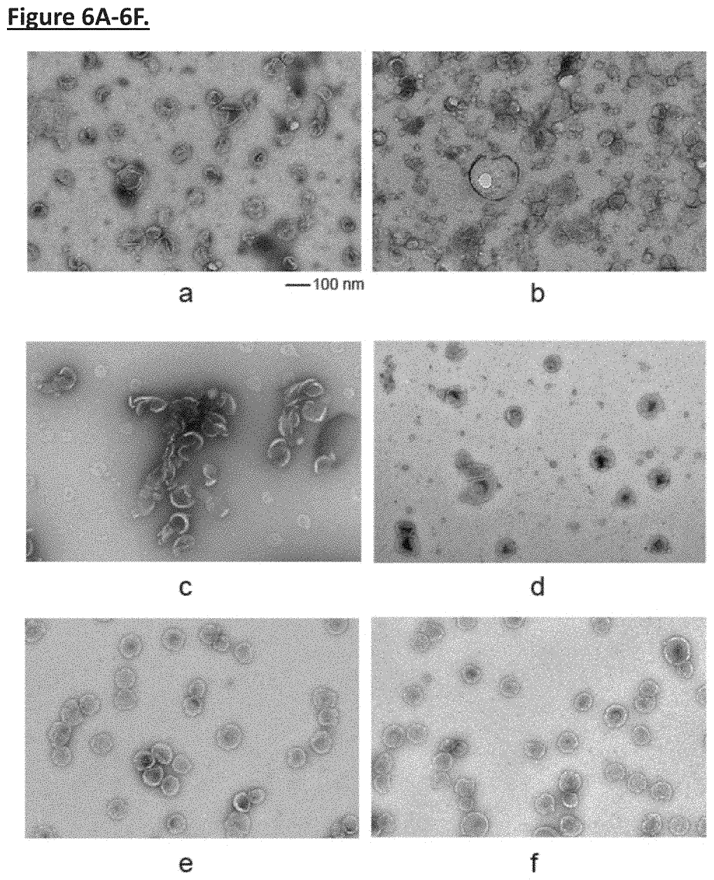

The Inventors introduced a His₆-tag at the C-terminus of the MPER-TMD to interact with the Ni-NTA-AuNPs. The MPER-TMD was expressed, purified, and reconstituted in bicelles with q=0.5 as described previously. The Ni-NTA-AuNP solution (OD530=0.1) and the solution of bicelle-reconstituted MPER-TMD-His₆ (0.3 mM) were mixed at a volume ratio of 3:1 to allow coating of protein-containing bicelles onto the nanoparticle surface (FIG. 2c). After complete removal of DH6PC by dialysis, relatively uniformly sized liposomes were formed around the nanoparticles (FIG. 2d, 2e). The diameter of the liposomes is 75±5 nm, which is consistent with the predicted size of the complex, including the Ni-NTA-AuNP (˜50 nm), the lipid bilayer (˜6 nm×2), and the space between Ni-NTA and His₆-tag, the Inventors performed a series of control experiments, i.e., forming liposomes 1) with empty bicelles in the absence of AuNPs (FIG. 6a), 2) by mixing Ni-NTA-AuNPs with empty bicelles nm), the lipid bilayer (˜6 nm×2), and the space between Ni-NTA-AuNP and lipid envelope. To provide additional evidence that the formation of stable nanoparticle-supported liposome is due to specific interaction between Ni-NTA and His₆-tag, the Inventors performed a series of control experiments, i.e., forming liposomes 1) with empty bicelles in the absence of AuNPs (FIG. 6a), 2) by mixing Ni-NTA-AuNPs with empty bicelles (FIG. 6b), and 3) by mixing non-functionalized AuNPs with bicelle-reconstituted MPER-TMD (FIG. 6c). As shown by negative staining EM (nsEM), liposomes formed under these conditions were mostly broken and highly inhomogeneous, indicating that direct recruitment of bicelles onto the nanoparticles via the membrane protein affinity tag is crucial to achieving homogeneous proteoliposome assemblies.

To achieve the highest proteoliposome assembly efficiency, different ratios of Ni-NTA-AuNP to MPER-TMD-His₆ were tested. When the volume ratio between the solution of Ni-NTA-AuNP (OD530=0.1) and the solution of bicelle-reconstituted MPER-TMD-His₆ (0.3 mM) was set at 1:1, in addition to the expected liposome size (˜75 nm), much smaller liposomes (<20 nm) were observed (FIG. 6d), likely formed with excessive MPER-TMD-His₆ and DMPC lipid without the support of Ni-NTA-AuNP. When the ratio was set to 3:1 and 6:1, the smaller liposomes mostly disappeared and very similar liposome populations were observed (FIG. 6e, 6f), suggesting that all protein-containing bicelles were recruited to Ni-NTA-AuNPs at the two ratios. Excessive Ni-NTA-AuNPs could not be observed due to incompatibility with negative staining.

The Inventors next tested the use of hollow DNA nanoparticles to guide proteoliposome assembly. The Inventors used a previously designed DNA buckyball formed with three different DNA strands (long, medium, and short). To functionalize the DNA buckyballs with NTA moieties, the Inventors used the TriNTA with modified primary amine, which can be covalently linked to thiol-modified DNA via an amine-to-sulfhydryl crosslinker (FIG. 7a). As such, the long strand was synthesized with the dithiol group at the 3′ end, and TriNTA was covalently linked to the long strand via a bifunctional crosslinker, PEG2-SMCC (Experimental Procedures). When charged with Ni²⁺, the TriNTA has high binding affinity for the His₆-tag (20±10 nM). The TriNTA-linked DNA strand was purified and mixed with the other two strands to form DNA buckyballs with an average diameter of ˜80 nm (FIG. 8b).

As in the AuNP application above, the bicelle-reconstituted MPER-TMD (with C-terminal His₆-tag) was mixed with the DNA buckyballs, and the ratio of MPER-TMD trimer to TriNTA (or long strand) was kept approximately at 1:1 to achieve ˜60 MPERTMD trimers per buckyball. Upon removal of DH6PC, spherical liposomes with diameter of 95±15 nm were formed (FIG. 2f, 9). The Inventors note that a fraction of liposomes are smaller than estimated size, probably because some DNA buckyballs were not completely assembled (thus smaller size) but could still catalyze liposome formation. More robust buckyball assembly can be achieved by using additional supportive DNA strands. To examine whether the MPER-TMD was unidirectionally presented on Ni-NTA-AuNP-supported liposomes, the Inventors introduced a FLAG-tag and a His₆-tag at the N- and C-termini of the MPER-TMD, respectively, and prepared Ni-NTA-AuNP supported proteoliposomes using the same protocol as described above.

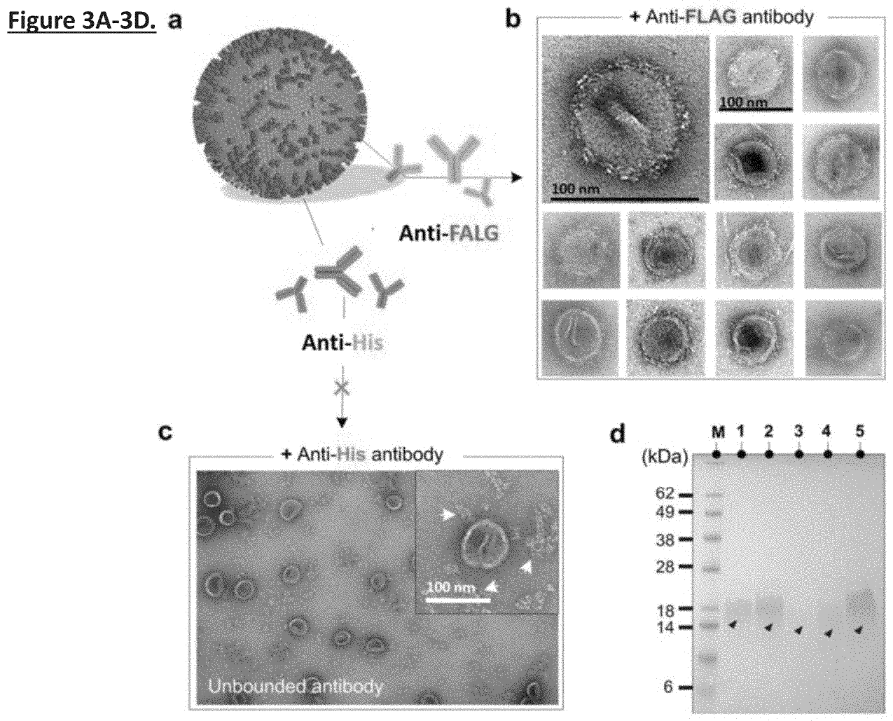

Anti-FLAG and anti-His₆ antibodies were mixed with the Ni-NTA-AuNP supported proteoliposomes separately (FIG. 3a) and visualized by nsEM. As expected, the anti-FLAG antibodies appeared to rest on the surface of the proteoliposomes (FIG. 3b), suggesting that the MPER-TMD N-termini are mostly exposed and accessible by the antibodies. In contrast, the anti-His₆ antibodies appeared randomly distributed around the liposomes and showed no obvious affinity to the liposome (FIG. 3c), suggesting that the MPER-TMD C-termini are mostly buried inside the liposome. In addition to the low-resolution images, the Inventors examined antibody binding more directly by performing antibody retaining experiments. Equal amount of reconstituted Ni-NTA-AuNP supported proteoliposomes were incubated with anti-FLAG and anti-His₆ resins separately. The flowthrough from the resins was collected as readout of MPER-TMDs that do not bind the antibodies. Further, a 0.1 M glycine solution was used to elute the resin-bound fraction. The samples from flow-through and elution were analyzed by SDS-PAGE. For the anti-His₆ resin, almost all MPER-TMDs were found in the flow-through (FIG. 3d; lanes 2, 3), whereas more than 90% of the MPER-TMDs were retained by the anti-FLAG resin (FIG. 3d; lanes 4, 5). The results indicate that the MPER-TMD was incorporated in the nanoparticle-supported liposomes in a unidirectional manner. More specifically, in the anti-His6 resin, 97.1% of MPER-TMDs were found in the flow-through, whereas 94.8% of the MPER-TMDs were retained by the anti-FLAG resin. The results indicate that the MPER-TMD was incorporated in the nanoparticle-supported liposomes in a unidirectional manner.

The Inventors have shown that nanoparticles with functionalized surfaces can serve as effective guide for the formation of proteoliposomes with unidirectional presentation of membrane proteins. Since the protein affinity tag drives uniform coating of bicelles, which are essentially solubilized membrane patches, around the nanoparticles, proteoliposome formation upon detergent removal is highly robust. Indeed, the EM images showed essentially no deformed liposomes. Moreover, varying sizes of proteoliposomes are achievable for different applications, as the size of the nanoparticle substrate can be accurately controlled.

The Inventors believe the nanoparticle-supported liposomes can be effective vaccine carriers. First, potentially high copy number of membrane proteins can be incorporated. The unidirectional presentation further increases the amount of effective antigens for the immune system. Second, the AuNP used in the current study is highly biocompatible and inexpensive to produce. The presence of nickel inside the liposome may be a safety concern but its toxicity is expected to be greatly reduced when chelated by NTA. Finally, the nanoparticle-supported liposome is structurally more stable than the regular liposomes owing to the nanoparticle-protein interaction, and such enhanced stability is important for application in vivo.

Previous attempts at presenting MPER in immunogens have not been successful in inducing neutralizing antibodies in vivo. The failure could be due to the conformation nature of the epitopes or their limited accessibility on the membrane surface. Recent studies suggest lipid bilayer also accounts for the neutralizing potency of MPER-specific antibodies. The reported method allowed unidirectional presentation of many MPER-TMD trimers on a single particle in a lipid bilayer environment. Indeed, the new immunogen elicited MPER-specific antibodies in the guinea pigs, though the neutralizing potential of these antibodies remains to be investigated.

In conclusion, the use of functionalized nanoparticles to guide proteoliposome formation offers many distinct advantages, including the improved efficiency and uniformity of liposome formation, the unidirectional presentation of transmembrane proteins, the preservation of membrane protein native structure, the greater control of protein incorporation number per liposome, and the greater stability of the nanoparticle-supported liposomes. While these advantages are particularly important for vaccine development, they are equally useful for developing therapeutic antibodies against membrane proteins such as GPCRs, transporters, and ion channels.

### Example 10

### Animal Data

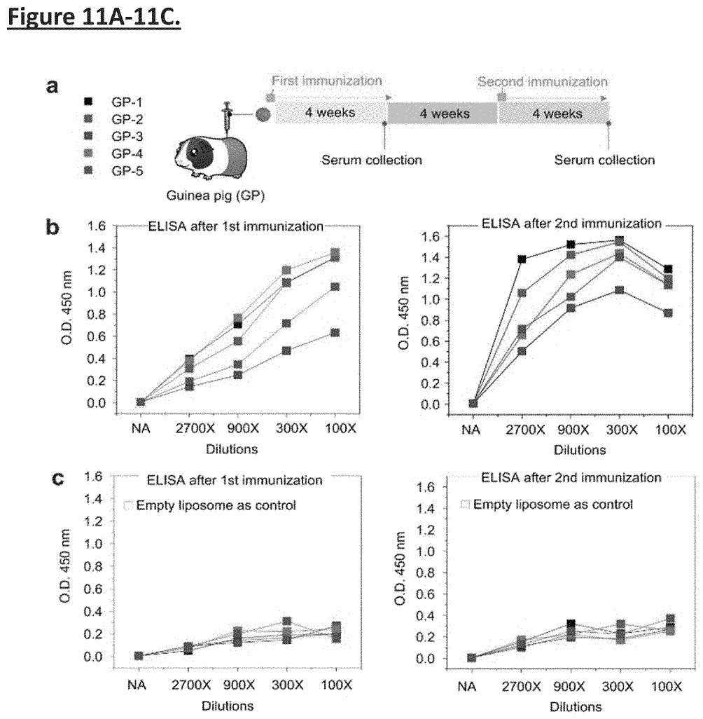

In further investigation, the Inventors tested whether the nanoparticle-supported proteoliposomes are immunogenic, five guinea pigs were immunized with Ni-NTA-AuNP-supported MPER-TMD proteoliposome with Adju-Phos adjuvant. Animal sera from different time points (FIG. 11a) were used to assess the ability of the vaccination regimen to elicit antibodies that can bind MPER-TMD reconstituted in regular liposome. The results from the enzyme-linked immunosorbent assay (ELISA) show that all of the sera after the first vaccination already contained MPER-TMD binding antibodies, and that sera from the second immunization elicited antibodies that bind much stronger and more robust to MPER-TMD (FIG. 11b). As a negative control, no binding to the empty liposome was observed (FIG. 11c).

### Example 11

### Antibody Potency Study

The Inventors then began to investigate whether membrane protein antigen, displayed on nanoparticle-supported liposomes using the aforementioned technology can induce stronger antibody production in vivo than those displayed on conventional liposomes.

For this study, the membrane protein antigen is a fragment of the HIV-1 envelope glycoprotein including the membrane-proximal external region (MPER) and the transmembrane domain (TMD), designated MPER-TMD. The structure of the MPER-TMD in its prefusion state in lipid bilayer was determined recently by the Inventors in, Fu et al, PNAS 2018; 115(38):E8892-E8899, which is incorporated by reference herein. Of note, in the HIV vaccine field, the MPER is known for being not very immunogenic. To compare in vivo immunogenicity of MPER-TMD presented by nanoparticle-supported liposome and by conventional liposome, the Inventors prepared 0.2 mg of MPER-TMD with a C-terminal His6Tag, reconstituted in DMPC-DHPC bicelles with [DMPC]/[DHPC] ratio (or the q ratio) of 0.5. The sample was then split into two halves for the preparation of two test articles used for mouse immunization.

### Example 12

### Antibody Potency Study—Study Design

Test Article 1: The bicelle reconstituted MPER-TMD-His6Tag was mixed with Ni-NTA-functionalized, polyphenol-stabilized gold nanoparticle (AuNP), followed by dialysis against 25 mM phosphate buffer (pH 7.2, 50 mM NaCl) to remove the DHPC detergent. The resulting product is AuNP-supported proteoliposomes with MPER-TMD unidirectionally presented on the liposome surface.

Test Article 2: The bicelle reconstituted MPER-TMD-His6Tag was simply dialyzed against 25 mM phosphate buffer (pH 7.2, 50 mM NaCl) to remove the DHPC detergent to form multilamellar proteoliposomes. The sample was further processed with mini-extruder (Avanti Polar Lipids) to produce unilamellar proteoliposomes. The resulting product is conventional proteoliposomes with MPER-TMD randomly presented on the liposome surface.

### Example 12

### Mouse Immunization

Mouse immunizations and blood sampling were carried out under a contract by Covance (Denver, Pa., USA). Two mice groups (n=4/group) were used, immunized intraperitoneally with Test Article 1 and Test Article 2, respectively, at weeks 0, 3, and 6. In both cases, each immunization consisted of a total of 5 μg of MPER-TMD per injection, formulated in 50% Imject Alum as adjuvent (Thermo Scientific, USA). Serum samples were collected 11 days after each immunization.

Animal sera collected from different time points were used to assess the ability of the immunization regimen to elicit antibodies that can bind MPER-TMD, and this was measured using an enzyme-linked immunosorbent assay (ELISA). The antigen used for the ELISA was an MPER-TMD proteoliposome sample prepared in the same way as for Test Article 2 above. ELISA plates (Thermo Scientific, USA) were coated with MPER-TMD proteoliposomes and incubated overnight. Mouse sera were then added in serial dilutions (100, 300, 900, and 2700) and detected with a horseradish peroxidase (HRP)-conjugated rabbit anti-guinea pig secondary antibody for total IgG ELISAs (Thermo Fisher). Plates were developed and read using the Emax Precision Microplate reader (Molecular Devices) at 450 nm.

### Example 13

### Results

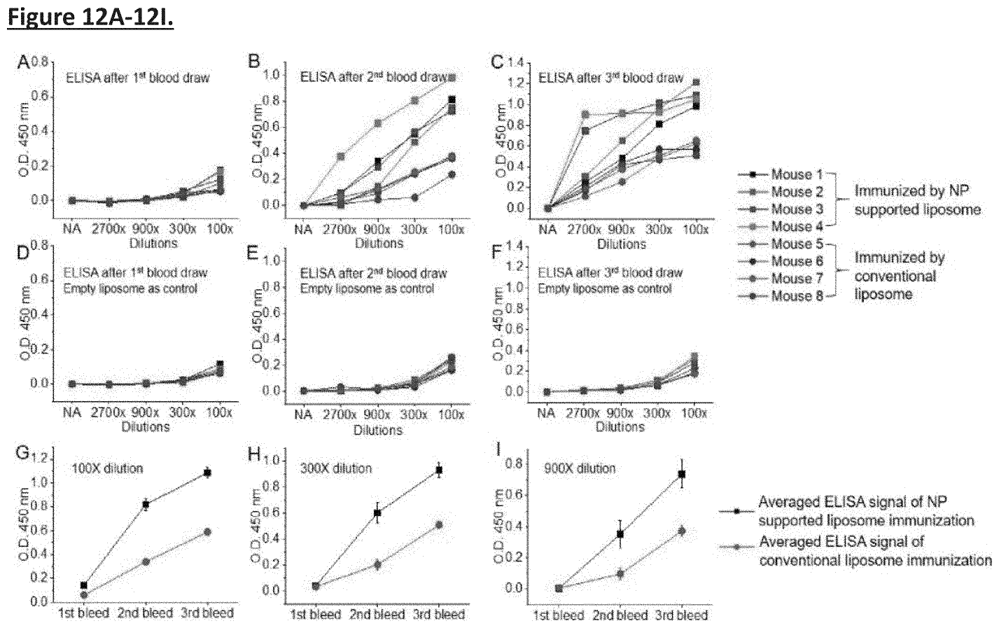

The ELISA results show that all of the sera after the first immunization with AuNP-supported proteoliposomes already contained anti-MPER-TMD antibodies (FIG. 12A; squares), and that sera from the second and third immunization elicited antibodies that bind much stronger and more robust to MPER-TMD (FIG. 12B and FIG. 12C; squares). For conventional proteoliposomes, the sera from first immunization showed almost no specific ELISA signal against MPER-TMD (FIG. 12A; circles). Although sera from the second and the third immunization induced more MPER-TMD specific antibodies (FIG. 12B and FIG. 12C; circles), their ELISA signal intensity are only 30-50% of those from the corresponding immunization using the AuNP-supported proteoliposomes. As a negative control, no strong binding to the empty liposomes was observed (FIG. 12D, FIG. 12E, and FIG. 12F). The ELISA signals averaged over 4 mice in each group show that anti-MPER-TMD antibody production induced by the AuNP-supported proteoliposomes is at least twofold stronger than that induced by conventional proteoliposomes for all dilution factors (FIG. 12G, FIG. 12H, and FIG. 12I).

### Example 14

### Conclusion

Using the same amount of immunogens, we found that the nanoparticle-supported proteoliposomes as the antigen presentation vehicles induced at least two-fold stronger specific antibody response than that induced by conventional proteoliposomes. The greater immunogenicity of the nanoparticle-supported proteoliposomes could be due to either unidirectional presentation of the MPER-TMD, which effectively increases the amount of exposed MPER, or the much higher stability of the supported proteoliposomes in vivo. Given the extremely low production cost of AuNP, we believe the AuNP-supported liposomes provides substantial benefit over the conventional liposomes as vaccine delivery vehicles.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are techniques and compositions for generating nanoparticles, including proteoliposome coated nanoparticles for unidirectional presentation of membrane proteins, including transmembrane proteins, manufacturing techniques for such nanoparticles, proteoliposomes and membrane proteins used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. An assembly comprising:
a substrate,
a coating, and
a quantity of lipids comprising a protein with a tag, wherein the coating is attached to the substrate and bound to the tag.

2.-3. (canceled)

4. The assembly of claim 1, wherein the substrate is a nanoparticle.

5. The assembly of claim 4, wherein the nanoparticle comprises a gold nanoparticle, silver nanoparticle, platinum nanoparticle, silicon dioxide nanoparticle, porous silicon nanoparticle, polymer nanoparticle, and/or complex nanoparticle.

6. The assembly of claim 1, wherein the substrate is a biological molecule, wherein the biological molecule is selected from the group consisting of: DNA, RNA, PNA, LNA, and protein.

7. (canceled)

8. The assembly of claim 6, wherein the DNA is a buckyball, cube, tetrahedron, dodecahedron, pyramid, tube, or stick.

9. The assembly of claim 6, wherein the DNA comprises a di-sulfide modifier, amino modifier, azide modifier, acrydite modifier, alkyne modifier, biotin, and/or digoxigenin.

10. The assembly of claim 1, wherein the coating comprises polyphenol, tannic acid, catechin, dopamine, theaflavin, anthocyanidin, and derivatives thereof.

11. The assembly of claim 1, wherein the coating comprises one or more molecules selected from the group consisting of: PEG-SMCC, AMAS, BMPS, GMBS, MBS, EMCS, SMPB, SMPH, SPDP, and SMPT.

12. The assembly of claim 1, wherein the coating comprises lysine, and/or cysteine.

13. The assembly of claim 1, wherein the coating comprises NTA-Ni, antibodies, nanobodies, biotin, and/or streptavidin

14. The assembly of claim 1, wherein the lipids are bicelles, synthetic lipids, extracts from host, and combinations thereof.

15. The assembly of claim 1, wherein the tag is one or more tags selected from the group consisting of: histidine, E tag, calmodulin tag, Myc tag, NE tag, S tag, SBP tag, Strep tag, Spot tag, pilin-C tag, Flag tag, HA tag, TC tag, Ty tag, V5 tag, and VSV tag.

16. The assembly of claim 1, wherein the coating attached to the substrate and bound to the tag externally presents a feature of the protein.

17.-33. (canceled)

34. A method of detecting a protein, comprising:
incubating a tagged protein with a lipid and a detergent to form an assembly;
removing the detergent;
incubating the assembly in the presence of a coated substrate, wherein the coated substrate comprises a functional moiety capable of binding to the tagged protein;
forming proteoliposomes on the coated substrate to externally present a feature of the tagged protein; and
detecting the protein using the externally presented feature.

35. The method of claim 34, wherein the protein is a membrane bound protein, including transmembrane protein, membrane transport protein, channel protein, membrane receptor, membrane anchored protein, and membrane protein complex.

36. A nanoparticle vaccine comprising:
a nanoparticle substrate,
a coating, and
a quantity of lipids comprising a microbial protein with a tag, wherein the coating is attached to the nanoparticle substrate and bound to the tag.

37. The nanoparticle vaccine of claim 36, wherein the nanoparticle substrate is a gold nanoparticle (AuNP) and the coating comprises polyphenol and NTA-Ni.

38. The nanoparticle vaccine of claim 36, wherein the microbial protein comprises a Human Immunodeficiency Virus (HIV) transmembrane protein.

39. The nanoparticle vaccine of claim 38, wherein the HIV transmembrane protein comprises the membrane-proximal external region (MPER) and the transmembrane domain (TMD) of HIV-1 envelope glycoprotein gp1, and wherein the HIV transmembrane protein is linked to a histidine tag.

40. A method of immunizing against a microbial infection comprising administering an effective amount of the nanoparticle vaccine of claim 36 to a subject in need thereof.


SEQUENCE LISTING (3 sequences)

SEQ ID NO: 1 - DNA, 72 bp, Artificial Sequence
  Bucky Long
aggcaccatcgtaggtttcttgccaggcaccatcgtaggtttcttgccaggcaccatcgtaggtttcttgcc

SEQ ID NO: 2 - DNA, 42 bp, Artificial Sequence
  Bucky Medium
tagcaacctgcctggcaagcctacgatggacacggtaacgcc

SEQ ID NO: 3 - DNA, 21 bp, Artificial Sequence
  Bucky Short
ttaccgtgtggttgctaggcg